(19) United States
(12) Patent Application Publication
(10) Pub. No.: US 2026/0263837 A1
(43) Pub. Date: Sep. 10, 2026

(54) CHERENKOV-BASED CONTROL OF A RADIATION TREATMENT APPARATUS

(71) Applicant: Brainlab SE, Munich (DE)

(72) Inventors: Hagen Kaiser, Munich (DE); Kajetan BERLINGER, Munich (DE)

(21) Appl. No.: 19/166,330

(22) PCT Filed: Apr. 26, 2023
(86) PCT No.: PCT/EP2023/060976

§ 371 Date: Sep. 17, 2025

(51) Int. Cl. A61N 5/10; G16H 20/40

(52) U.S. Cl. CPC A61N 5/1039; A61N 5/1037; G16H 20/40

(57) ABSTRACT

Disclosed is a computer-implemented method of determining control data for controlling a radiation treatment apparatus. The disclosed method encompasses, in one aspect, acquisition of a Cherenkov radiation image from a body surface of a patient and comparing it to a simulated Cherenkov radiation pattern such as for example with regard to its position relative to an anatomical body part which is designated to be irradiated with treatment radiation. The result of the comparison is used to control a radiation treatment apparatus, such as for example to switch the beam off if the comparison indicates that the position of the detected Cherenkov radiation relative to the anatomical body part is not according to plan.

### FIELD OF THE INVENTION

The present invention relates to computer-implemented methods of determining control data for controlling a radiation treatment apparatus, a corresponding computer program, a computer-readable storage medium storing such a program and a computer executing the program, as well as a medical system comprising an electronic data storage device and the aforementioned computer.

### TECHNICAL BACKGROUND

Known approaches using a Cherenkov camera to infer applied radiation dose by texture rendering the Cherenkov images onto a 3D model of the patient do not take into account the current position of the patient to the radiation treatment apparatus. Therefore, a live monitoring 3D camera is required to detect that position which results in latency due to the need for creating a wire mesh out of the point cloud generated by the 3D camera. It is desirable to remove such a latency when controlling a radiation treatment apparatus.

The present invention has the object of providing an improved method of controlling a radiation treatment apparatus.

The present invention can be used for procedures e.g. in connection with a system for image-guided radiotherapy such as VERO® and ExacTrac®, both products of Brainlab AG.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The disclosed method encompasses, in one aspect, acquisition of a Cherenkov radiation image from a body surface of a patient and comparing it to a simulated Cherenkov radiation pattern for example with regard to its position relative to an anatomical body part which is designated to be irradiated with treatment radiation. The result of the comparison is used to control a radiation treatment apparatus, for example to switch the beam off if the comparison indicates that the position of the detected Cherenkov radiation relative to the anatomical body part is not according to plan.

### GENERAL DESCRIPTION OF THE INVENTION

In this section, a description of the general features of the present invention is given for example by referring to possible embodiments of the invention.

In general, the invention reaches the aforementioned object by providing, in a first aspect, a computer-implemented medical method of determining control data for controlling a radiation treatment apparatus. The method according to the first aspect comprises executing, on at least one processor of at least one computer (for example at least one computer being part of a navigation system), the following exemplary steps which are executed by the at least one processor.

In a (for example first) exemplary step of the method according to the first aspect, radiation pattern image data is acquired which describes a digital image describing a Cherenkov radiation pattern on an anatomical body part of patient, for example of a surface of a patient's body, taken with an imaging device, a relative position between the imaging device and the radiation treatment apparatus being predetermined, for example known to the method. The Cherenkov radiation pattern is created as a result of irradiating the anatomical body part with ionising radiation.

In a (for example second) exemplary step of the method according to the first aspect, simulated radiation pattern data is acquired which describes a simulated Cherenkov radiation pattern on the anatomical body part which has been simulated based on a planning image, for example a contour determined from a computed x-ray tomography, of the anatomical body part. The simulated Cherenkov radiation pattern is created as a result of simulating irradiation of the anatomical body part with ionising radiation.

In a (for example third) exemplary step of the method according to the first aspect, pattern comparison data is determined based on the radiation pattern image data and the simulated radiation pattern data, wherein the pattern comparison data describes a pattern comparison result describing a result of comparing the Cherenkov radiation pattern described by the digital image to the simulated Cherenkov pattern. For example, the comparison result describes the similarity of the Cherenkov radiation pattern described by the digital image and the simulated Cherenkov pattern, for example the similarity of the position of the Cherenkov radiation pattern described by the digital image relative to the anatomical body part and the position of the simulated Cherenkov pattern relative to the anatomical body part.

In a (for example fourth) exemplary step of the method according to the first aspect, threshold data is acquired which describes a predetermined threshold for the pattern comparison result. For example, the predetermined threshold is a threshold for the similarity of the Cherenkov radiation pattern described by the digital image and the simulated Cherenkov pattern, for example the similarity of the position of the Cherenkov radiation pattern described by the digital image relative to the anatomical body part and the position of the simulated Cherenkov pattern relative to the anatomical body part.

In a (for example fifth) exemplary step of the method according to the first aspect, treatment apparatus control data is determined based on the pattern comparison data and the threshold data, wherein the treatment apparatus control data is determined by comparing the pattern comparison result to the predetermined threshold and describes a control signal to be issued to the radiation treatment apparatus. For example, apparatus control data is determined, for example the control signal is generated if the pattern comparison result fulfils a predetermined condition relative to the predetermined threshold, for example is equal to or greater than the predetermined threshold.

In an example of the method according to the first aspect, planning image data is acquired which describes the planning image and a relative position between positions described by the planning image data and the position of the radiation treatment apparatus. For example, the position of the radiation treatment apparatus is predetermined.

In an example of the method according to the first aspect, surface image data is acquired which describes a surface image of the anatomical body part. The surface image data has been generated using a surface-generating device, for example a surface camera, wherein a relative position between the surface-generating device and the radiation treatment apparatus is predetermined. In this example, transformation data is determined based on the planning image data and the surface image data, wherein the transformation data describes a spatial transformation (for example, a mapping) between positions described by the surface image data and positions described by the planning image data. Also, deformed planning image data is determined based on the planning image data and the surface image data, wherein the deformed planning image data describes a deformed planning image which is determined by applying the spatial transformation to the planning image. The simulated radiation pattern data is then determined based on the deformed planning image data, for example by generating the simulated Cherenkov pattern on the deformed planning image data. The spatial transformation is determined for example by applying an elastic fusion algorithm to the surface image data and the planning image data.

The invention relates, in a second aspect, to a computer-implemented medical method of determining control data for controlling a radiation treatment apparatus. The method according to the second aspect comprises executing, on at least one processor of at least one computer (for example at least one computer being part of a navigation system), the following exemplary steps which are executed by the at least one processor.

In a (for example first) exemplary step of the method according to the second aspect, planning image data is acquired which describes a planning image of the anatomical body part, wherein a relative position between positions described by the planning image data and the position of the radiation treatment apparatus is predetermined.

In a (for example second) exemplary step of the method according to the second aspect, first simulated radiation pattern data is acquired. The first simulated radiation pattern data describes a first simulated Cherenkov radiation pattern on the anatomical body part which has been simulated based on the planning image. The first simulated Cherenkov radiation pattern is created as a result of simulating irradiation of the anatomical body part with ionising radiation.

In a (for example third) exemplary step of the method according to the second aspect, surface image data is acquired which describes a surface image of the anatomical body part generated using a surface-generating device, wherein a relative position between the surface-generating device and the radiation treatment apparatus is predetermined.

In a (for example fourth) exemplary step of the method according to the second aspect, transformation data is determined based on the planning image data and the surface image data. The transformation data describes a spatial transformation between positions described by the surface image data and positions described by the planning image data. For example, the transformation data is determined by establishing the spatial transformation.

In a (for example fifth) exemplary step of the method according to the second aspect, deformed planning image data is determined based on the planning image data and the surface image data, wherein the deformed planning image data describes a deformed planning image of the anatomical body part which is determined by applying the spatial transformation to the planning image.

In a (for example sixth) exemplary step of the method according to the second aspect, second simulated radiation pattern data is determined based on the deformed planning image data. The second simulated radiation pattern data describes a second simulated Cherenkov radiation pattern on the anatomical body part which has been simulated based on the deformed planning image. The second simulated Cherenkov radiation pattern is created as a result of simulating irradiation of the anatomical body part with ionising radiation.

In a (for example seventh) exemplary step of the method according to the second aspect, simulation comparison data is determined based on the first simulated radiation pattern data and the second simulated radiation pattern data, wherein the simulation comparison data describes a simulation comparison result describing a result of comparing the first simulated Cherenkov radiation pattern and the second simulated Cherenkov radiation pattern. For example, the simulation comparison result describes the similarity of the first simulated Cherenkov radiation pattern and the second simulated Cherenkov radiation pattern, for example the similarity of the position of the first simulated Cherenkov radiation pattern relative to the anatomical body part and the position of the second simulated Cherenkov pattern relative to the anatomical body part.

In a (for example eighth) exemplary step of the method according to the second aspect, threshold data is acquired which describes a predetermined threshold for the simulation comparison result. For example, the predetermined threshold is a threshold for the similarity of the first simulated Cherenkov radiation pattern and the second simulated Cherenkov radiation pattern, for example the similarity of the first simulated Cherenkov radiation pattern relative to the anatomical body part and the position of the second simulated Cherenkov pattern relative to the anatomical body part.

In a (for example ninth) exemplary step of the method according to the second aspect, treatment apparatus control data is determined based on the simulation comparison data and the threshold data. The treatment apparatus control data is determined by comparing the simulation comparison result to the predetermined threshold and describes a control signal to be issued to the radiation treatment apparatus. For example, apparatus control data is determined, for example the control signal is generated if the pattern comparison result fulfils a predetermined condition relative to the predetermined threshold, for example is equal to or greater than the predetermined threshold.

In examples of the methods according to the first and second aspects, the control signal describes a command for stopping beam emission by the radiation treatment apparatus.

In examples of the methods according to the first and second aspects, the predetermined threshold is defined as at least one of the following:
a sum of squared differences,
a correlation coefficient,
a normalized cross correlation, or
mutual information
   between the Cherenkov radiation pattern and the simulated Cherenkov radiation pattern or the first simulated Cherenkov radiation pattern and the second simulated Cherenkov radiation pattern, respectively.

In examples of the methods according to the first and second aspects, the predetermined threshold is defined as at least one of the following:
a tessellation of the Cherenkov radiation pattern and the simulated Cherenkov radiation pattern or the first simulated Cherenkov radiation pattern and the second simulated Cherenkov radiation pattern, respectively;
matching corresponding subimages of the Cherenkov radiation pattern and the simulated Cherenkov radiation pattern or the first simulated Cherenkov radiation pattern and the second simulated Cherenkov radiation pattern, respectively;
visually marked squares that yield a deviation of the Cherenkov radiation pattern from a reference position defined by the simulated Cherenkov radiation pattern or a deviation of the first simulated Cherenkov radiation pattern from a reference position defined by the second simulated Cherenkov radiation pattern, respectively.

In examples of the methods according to the first and second aspects, the planning image has been selected based on breathing phase data describing a breathing phase of the patient, for example in accordance with a predetermined breathing phase. For example, the methods according to the first and second aspects comprise acquiring the breathing phase data. For example, the breathing phase data has been generated based on detecting a position of a marker device having a predetermined position relative to the patient's body, a strain signal generated by a strain gauge attached to the patient's body, or a thermal camera generating a surface image of at least part of the patient's thorax.

The method according to any one of the preceding claims, wherein the planning image has been generated using a tomographic imaging modality, for example a computed x-ray tomography imaging modality or a magnetic resonance imaging modality or ultrasound imaging modality.

In a third aspect, the invention is directed to a computer program comprising instructions which, when the program is executed by at least one computer, causes the at least one computer to carry out method according to the first aspect or the second aspect. The invention may alternatively or additionally relate to a (physical, for example electrical, for example technically generated) signal wave, for example a digital signal wave, such as an electromagnetic carrier wave carrying information which represents the program, for example the aforementioned program, which for example comprises code means which are adapted to perform any or all of the steps of the method according to the first aspect. The signal wave is in one example a data carrier signal carrying the aforementioned computer program. A computer program stored on a disc is a data file, and when the file is read out and transmitted it becomes a data stream for example in the form of a (physical, for example electrical, for example technically generated) signal. The signal can be implemented as the signal wave, for example as the electromagnetic carrier wave which is described herein. For example, the signal, for example the signal wave is constituted to be transmitted via a computer network, for example LAN, WLAN, WAN, mobile network, for example the internet. For example, the signal, for example the signal wave, is constituted to be transmitted by optic or acoustic data transmission. The invention according to the second aspect therefore may alternatively or additionally relate to a data stream representative of the aforementioned program, i.e. comprising the program.

In a fourth aspect, the invention is directed to a computer-readable storage medium on which the program according to the third aspect is stored. The program storage medium is for example non-transitory.

In a fifth aspect, the invention is directed to at least one computer (for example, a computer), comprising at least one processor (for example, a processor), wherein the program according to the third aspect is executed by the processor, or wherein the at least one computer comprises the computer-readable storage medium according to the fourth aspect.

In a sixth aspect, the invention is directed to a medical system, comprising:
a) the computer according the fifth aspect; and
b) a radiation treatment apparatus for carrying out radiation treatment on the patient,
   wherein the computer is operably coupled to the radiation treatment for issuing a control signal to the radiation treatment apparatus for controlling the operation of the radiation treatment apparatus on the basis of the treatment apparatus control data.

For example, the disclosed method is not a method for treatment of the human or animal body by surgery or therapy. For example, the invention does not involve or in particular comprise or encompass an invasive step which would represent a substantial physical interference with the body requiring professional medical expertise to be carried out and entailing a substantial health risk even when carried out with the required professional care and expertise.

For example, the invention does not comprise a step of irradiating the patient with ionizing radiation, for example treatment radiation. More particularly, the invention does not involve or in particular comprise or encompass any surgical or therapeutic activity. The invention is instead directed as applicable to processing acquired data.

For this reason alone, no surgical or therapeutic activity and in particular no surgical or therapeutic step is necessitated or implied by carrying out the invention.

### Definitions

In this section, definitions for specific terminology used in this disclosure are offered which also form part of the present disclosure.

The method in accordance with the invention is for example a computer-implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term computer includes a server resource. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a computer program comprising instructions which, when on the program is executed by a computer, cause the computer to carry out the method or methods, for example, the steps of the method or methods, described herein and/or to a computer-readable storage medium (for example, a non-transitory computer-readable storage medium) on which the program is stored and/or to a computer comprising said program storage medium and/or to a (physical, for example electrical, for example technically generated) signal wave, for example a digital signal wave, such as an electromagnetic carrier wave carrying information which represents the program, for example the aforementioned program, which for example comprises code means which are adapted to perform any or all of the method steps described herein. The signal wave is in one example a data carrier signal carrying the aforementioned computer program. The invention also relates to a computer comprising at least one processor and/or the aforementioned computer-readable storage medium and for example a memory, wherein the program is executed by the processor.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. A step of “determining” as described herein for example comprises or consists of issuing a command to perform the determination described herein. For example, the step comprises or consists of issuing a command to cause a computer, for example a remote computer, for example a remote server, for example in the cloud, to perform the determination. Alternatively or additionally, a step of “determination” as described herein for example comprises or consists of receiving the data resulting from the determination described herein, for example receiving the resulting data from the remote computer, for example from that remote computer which has been caused to perform the determination. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

Mapping describes a transformation (for example, linear transformation) of an element (for example, a pixel or voxel), for example the position of an element, of a first data set in a first coordinate system to an element (for example, a pixel or voxel), for example the position of an element, of a second data set in a second coordinate system (which may have a basis which is different from the basis of the first coordinate system). In one embodiment, the mapping is determined by comparing (for example, matching) the color values (for example grey values) of the respective elements by means of an elastic or rigid fusion algorithm. The mapping is embodied for example by a transformation matrix (such as a matrix defining an affine transformation).

Image fusion can be elastic image fusion or rigid image fusion. In the case of rigid image fusion, the relative position between the pixels of a 2D image and/or voxels of a 3D image is fixed, while in the case of elastic image fusion, the relative positions are allowed to change.

In this application, the term “image morphing” is also used as an alternative to the term “elastic image fusion”, but with the same meaning.

Elastic fusion transformations (for example, elastic image fusion transformations) are for example designed to enable a seamless transition from one dataset (for example a first dataset such as for example a first image) to another dataset (for example a second dataset such as for example a second image). The transformation is for example designed such that one of the first and second datasets (images) is deformed, for example in such a way that corresponding structures (for example, corresponding image elements) are arranged at the same position as in the other of the first and second images. The deformed (transformed) image which is transformed from one of the first and second images is for example as similar as possible to the other of the first and second images. Preferably, (numerical) optimisation algorithms are applied in order to find the transformation which results in an optimum degree of similarity. The degree of similarity is preferably measured by way of a measure of similarity (also referred to in the following as a “similarity measure”). The parameters of the optimisation algorithm are for example vectors of a deformation field. These vectors are determined by the optimisation algorithm in such a way as to result in an optimum degree of similarity. Thus, the optimum degree of similarity represents a condition, for example a constraint, for the optimisation algorithm. The bases of the vectors lie for example at voxel positions of one of the first and second images which is to be transformed, and the tips of the vectors lie at the corresponding voxel positions in the transformed image. A plurality of these vectors is preferably provided, for instance more than twenty or a hundred or a thousand or ten thousand, etc. Preferably, there are (other) constraints on the transformation (deformation), for example in order to avoid pathological deformations (for instance, all the voxels being shifted to the same position by the transformation). These constraints include for example the constraint that the transformation is regular, which for example means that a Jacobian determinant calculated from a matrix of the deformation field (for example, the vector field) is larger than zero, and also the constraint that the transformed (deformed) image is not self-intersecting and for example that the transformed (deformed) image does not comprise faults and/or ruptures. The constraints include for example the constraint that if a regular grid is transformed simultaneously with the image and in a corresponding manner, the grid is not allowed to interfold at any of its locations. The optimising problem is for example solved iteratively, for example by means of an optimisation algorithm which is for example a first-order optimisation algorithm, such as a gradient descent algorithm. Other examples of optimisation algorithms include optimisation algorithms which do not use derivations, such as the downhill simplex algorithm, or algorithms which use higher-order derivatives such as Newton-like algorithms. The optimisation algorithm preferably performs a local optimisation. If there is a plurality of local optima, global algorithms such as simulated annealing or generic algorithms can be used. In the case of linear optimisation problems, the simplex method can for instance be used.

In the steps of the optimisation algorithms, the voxels are for example shifted by a magnitude in a direction such that the degree of similarity is increased. This magnitude is preferably less than a predefined limit, for instance less than one tenth or one hundredth or one thousandth of the diameter of the image, and for example about equal to or less than the distance between neighbouring voxels. Large deformations can be implemented, for example due to a high number of (iteration) steps.

The determined elastic fusion transformation can for example be used to determine a degree of similarity (or similarity measure, see above) between the first and second datasets (first and second images). To this end, the deviation between the elastic fusion transformation and an identity transformation is determined. The degree of deviation can for instance be calculated by determining the difference between the determinant of the elastic fusion transformation and the identity transformation. The higher the deviation, the lower the similarity, hence the degree of deviation can be used to determine a measure of similarity.

A measure of similarity can for example be determined on the basis of a determined correlation between the first and second datasets.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 illustrates the basic steps of the method according to the first aspect;

FIG. 2 illustrates the basic steps of the method according to the second aspect;

FIG. 3 shows an embodiment of the present invention, specifically the methods according to the first and second aspects;

FIG. 4 is a schematic illustration of the system according to the sixth aspect.

### DESCRIPTION OF EMBODIMENTS

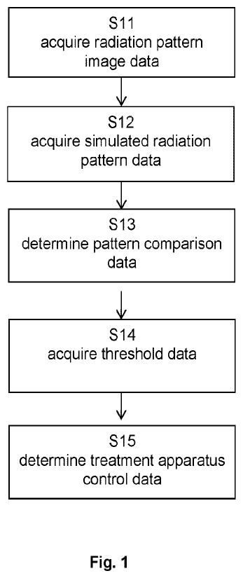

FIG. 1 illustrates the basic steps of the method according to the first aspect, in which step S11 encompasses acquisition of the radiation pattern image data, step S12 encompasses acquisition of the simulated radiation pattern data, step S13 is directed to determining the pattern comparison data and subsequent step S143 encompasses acquisition of the threshold data. In step S15, the treatment apparatus control data is determined based on the acquired data sets.

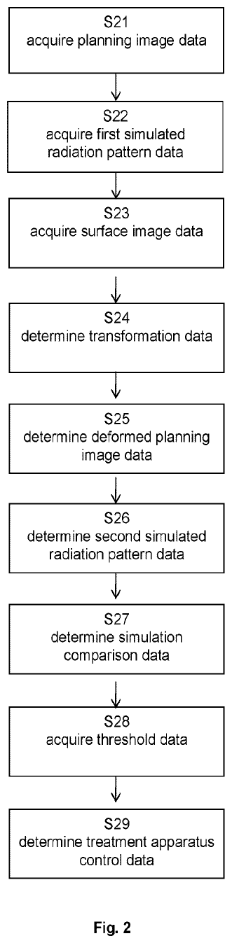

FIG. 2 illustrates the basic steps of the method according to the second aspect, in which step S21 encompasses acquisition of the planning image data, step S22 encompasses acquisition of the first simulated radiation pattern data, step S23 is directed to acquiring the surface image data, step S24 is directed to determining the transformation data, step S25 is directed to determining the deformed planning image data, step S26 encompasses determination of the second simulated radiation pattern data and subsequent steps S27 encompass acquisition of the threshold data and determination of the treatment apparatus control data, respectively.

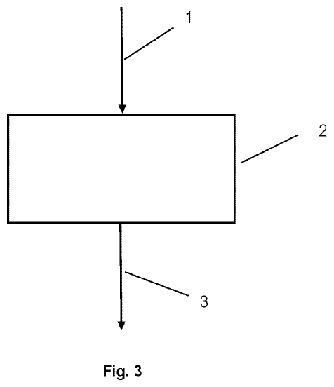

FIG. 3 illustrates an embodiment of the present invention that includes all essential features of the invention. In this embodiment, the entire data processing which is part of the method according to the first aspect is performed by a computer 2. Reference sign 1 denotes the input of data acquired by the method according to the first aspect into the computer 2 and reference sign 3 denotes the output of data determined by the method according to the first aspect.

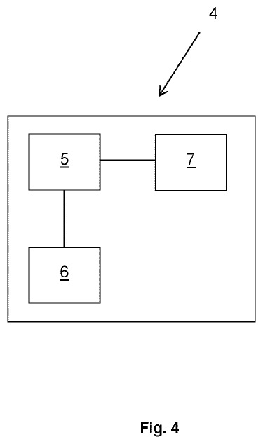

FIG. 4 is a schematic illustration of the medical system 4 according to the sixth aspect. The system is in its entirety identified by reference sign 4 and comprises a computer 5, an electronic data storage device (such as a hard disc) 6 for storing at least the patient data and a medical device 7 (such as a radiation treatment apparatus). The components of the medical system 4 have the functionalities and properties explained above with regard to the sixth aspect of this disclosure.

Embodiments of the invention are as follows:

### Embodiment 1

a) Acquire Cherenkov image from Cherenkov camera (1st camera).
b) Orientation (1st orientation) of Cherenkov camera to treatment device is known.
c) Acquire outer contour from planning CT from treatment plan.
d) Planned orientation (2nd orientation) from planning CT/outer Contour to treatment device is known from treatment plan.
e) Simulate Cherenkov image (1st simulated Cherenkov image) using planning CT/outer contour and 1st and 2nd orientation.
f) Cherenkov image is compared to simulated Cherenkov (1st simulated Cherenkov image) image.
g) Treatment is paused depending on predefined threshold.

### Embodiment 2, Dependent on Embodiment 1

a) Acquire surface of the patient from surface camera (2nd camera).
b) Orientation (3rd orientation) of surface camera to treatment device is known.
c) Acquire surface image of the patient from surface camera.
d) Acquire outer contour from planning CT from treatment plan.
e) Perform elastic fusion/deform planning CT in a way to match the surface image of the patient (=adapted CT/outer contour).
f) Simulate Cherenkov image (1st simulated Cherenkov image) using Adapted CT/Outer Contour and 1st and 2nd orientation.
g) Cherenkov image is compared to simulated Cherenkov image (1st simulated Cherenkov image).
h) Treatment is paused depending on predefined threshold.

### Embodiment 3, Dependent on Embodiment 1

i) Acquire surface of the patient from surface camera (2nd camera).
j) Orientation (3rd orientation) of surface camera to treatment device is known.
k) Acquire surface image of the patient from surface camera.
l) Acquire outer contour from planning CT from treatment plan.
m) Perform elastic fusion/deform planning CT in a way to match the surface image of the patient (=Adapted CT/Outer Contour).
n) Simulate Cherenkov image (2nd simulated Cherenkov image) using adapted CT/outer contour and 1st and 2nd orientation.
o) 2nd simulated Cherenkov image is compared to 1st simulated Cherenkov image.
p) Automatic suggestion if treatment can be started depending on predefined threshold.

### Embodiment 4, Dependent on Embodiment 1

a) Acquire live breathing signal from signalling device, e.g. IR camera tracking markers, strain gauge (Anzai belt), thermal camera.
b) Acquire outer contours from 4D-CT from treatment plan.
c) Select outer contour/bin corresponding to current live breathing level=current outer contour/bin (4D-CT comprises several bins, a bin representing a respiratory state [a bin can be regarded a CT]).
d) Simulate Cherenkov image (1st simulated Cherenkov image) using current outer contour/bin and 1st and 2nd orientation.
e) Cherenkov image is compared to simulated Cherenkov image (1st simulated Cherenkov image)—the images corresponding to approximately the same respiratory state).
f) Treatment is paused depending on predefined threshold.

The predetermined threshold is one of:
a quantitative measure:
Sum of squared differences
Correlation coefficient
Normalized cross correlation
Mutual information
a qualitative measure:
Tesselation of the images to be compared in e.g. squares of equal size (=subimages)
Matching corresponding subimages
Visually mark squares that yield deviation from reference position

1. A computer-implemented medical method in a system including a computer having a processor and a data storage device operatively coupled with the processor for determining control data for controlling an associated radiation treatment apparatus, the method comprising:
acquiring, by the processor of the computer, radiation pattern image data, which describes a digital image describing a Cherenkov radiation pattern on an anatomical body part of patient taken with an imaging device, a relative position between the imaging device and the radiation treatment apparatus being predetermined;
acquiring, by the processor of the computer, simulated radiation pattern data which describes a simulated Cherenkov radiation pattern on the anatomical body part which has been simulated based on a planning image of the anatomical body part;
determining, by the processor of the computer, pattern comparison data is based on the radiation pattern image data and the simulated radiation pattern data, wherein the pattern comparison data describes a pattern comparison result describing a result of comparing the Cherenkov radiation pattern described by the digital image to the simulated Cherenkov pattern;
acquiring, by the processor of the computer, threshold data, which describes a predetermined threshold for the pattern comparison result; and
determining, by the processor of the computer, treatment apparatus control data based on the pattern comparison data and the threshold data, wherein the treatment apparatus control data is determined by the processor of the computer by comparing the pattern comparison result to the predetermined threshold and wherein the treatment apparatus control data describes a control signal to be issued to the radiation treatment apparatus for controlling an operation of the associated radiation treatment apparatus.

2. The method according to claim 1, further comprising acquiring, by the processor of the computer, planning image data is acquired (S12) which describes the planning image and a relative position between positions described by the planning image data and a predetermined position of the radiation treatment apparatus.

3. The method according to claim 2, further comprising:
acquiring, by the processor of the computer, surface image data describing a surface image of the anatomical body part generated using an associated surface-generating device, wherein a relative position between the surface-generating device and the radiation treatment apparatus is predetermined;
determining, by the processor of the computer, transformation data based on the planning image data and the surface image data, wherein the transformation data describes a spatial transformation between positions described by the surface image data and positions described by the planning image data; and
determined, by the processor of the computer, deformed planning image data based on the planning image data and the surface image data, wherein the deformed planning image data describes a deformed planning image which is determined by applying the spatial transformation to the planning image,
wherein the simulated radiation pattern data is determined based on the deformed planning image data.

4. A computer-implemented medical method in a system including a computer having a processor and a data storage device operatively coupled with the processor for determining control data for controlling an associated radiation treatment apparatus, the method comprising:
acquiring, by the processor of the computer, planning image data, which describes a planning image of the anatomical body part, wherein a relative position between positions described by the planning image data and the position of the radiation treatment apparatus is predetermined;
acquiring, by the processor of the computer, first simulated radiation pattern data, which describes a first simulated Cherenkov radiation pattern on the anatomical body part which has been simulated based on the planning image;
acquiring, by the processor of the computer, surface image data, which describes a surface image of the anatomical body part generated using a surface-generating device, wherein a relative position between the surface-generating device and the radiation treatment apparatus is predetermined;
determining, by the processor of the computer, transformation data based on the planning image data and the surface image data, wherein the transformation data describes a spatial transformation between positions described by the surface image data and positions described by the planning image data;
determining, by the processor of the computer, deformed planning image data based on the planning image data and the surface image data, wherein the deformed planning image data describes a deformed planning image of the anatomical body part which is determined by applying the spatial transformation to the planning image;
determining, by the processor of the computer, second simulated radiation pattern data based on the deformed planning image data, wherein the second simulated radiation pattern data describes a second simulated Cherenkov radiation pattern on the anatomical body part which has been simulated based on the deformed planning image;
determining, by the processor of the computer, simulation comparison data based on the first simulated radiation pattern data and the second simulated radiation pattern data, wherein the simulation comparison data describes a simulation comparison result describing a result of comparing the first simulated Cherenkov radiation pattern and the second simulated Cherenkov radiation pattern;
acquiring, by the processor of the computer, threshold data, which describes a predetermined threshold for the simulation comparison result; and
determining, by the processor of the computer, treatment apparatus control data based on the simulation comparison data and the threshold data, wherein the treatment apparatus control data is determined by comparing the simulation comparison result to the predetermined threshold and wherein the treatment apparatus control data describes a control signal to be issued to the radiation treatment apparatus for controlling an operation of the associated radiation treatment apparatus.

5. The method according to claim 4, wherein the determining the treatment apparatus control data comprises determining treatment apparatus control data that describes a control signal that describes a command for stopping beam emission by the associated radiation treatment apparatus.

6. The method according to claim 4, wherein the acquiring the threshold data comprises acquiring threshold data which describes a predetermined threshold defined as at least one of:
a sum of squared differences;
a correlation coefficient;
a normalized cross correlation; and/or
mutual information between the Cherenkov radiation pattern and the simulated Cherenkov radiation pattern or the first simulated Cherenkov radiation pattern and the second simulated Cherenkov radiation pattern, respectively.

7. The method according to claim 4, wherein the acquiring the threshold data comprises acquiring threshold data which describes a predetermined threshold defined as at least one of:
a tessellation of the Cherenkov radiation pattern and the simulated Cherenkov radiation pattern or the first simulated Cherenkov radiation pattern and the second simulated Cherenkov radiation pattern, respectively;
matching corresponding subimages of the Cherenkov radiation pattern and the simulated Cherenkov radiation pattern or the first simulated Cherenkov radiation pattern and the second simulated Cherenkov radiation pattern, respectively; and/or
visually marked squares that yield a deviation of the Cherenkov radiation pattern from a reference position defined by the simulated Cherenkov radiation pattern or a deviation of the first simulated Cherenkov radiation pattern from a reference position defined by the second simulated Cherenkov radiation pattern, respectively.

8. The method according to claim 4, wherein:
the acquiring the planning image data comprises acquiring planning image data which describes a planning image that has been selected based on breathing phase data describing a breathing phase of the patient, in accordance with a predetermined breathing phase.

9. The method according to claim 8, further comprising:
acquiring, by the processor of the computer, the breathing phase data.

10. The method according to claim 9, wherein the acquiring the breathing phase data comprises acquiring breathing phase data that has been generated based on detecting a position of a marker device having a predetermined position relative to the patient's body, a strain signal generated by a strain gauge attached to the patient's body, or a thermal camera generating a surface image of at least part of the patient's thorax.

11. The method according to claim 4, wherein the acquiring the planning image data comprises acquiring planning image data which describes a planning image that has been generated using a tomographic imaging modality, comprises a computed x-ray tomography imaging modality or a magnetic resonance imaging modality or ultrasound imaging modality.

12. A non-transitory computer-readable storage medium storing instructions thereon that, when the instructions are executed by a processor device of an associated computer, cause the computer to perform a method of determining control data for controlling an associated radiation treatment apparatus, the method comprising:
acquiring, by the processor of the computer, radiation pattern image data, which describes a digital image describing a Cherenkov radiation pattern on an anatomical body part of patient taken with an imaging device, a relative position between the imaging device and the radiation treatment apparatus being predetermined;
acquiring, by the processor of the computer, simulated radiation pattern data, which describes a simulated Cherenkov radiation pattern on the anatomical body part which has been simulated based on a planning image of the anatomical body part;
determining, by the processor of the computer, pattern comparison data is based on the radiation pattern image data and the simulated radiation pattern data, wherein the pattern comparison data describes a pattern comparison result describing a result of comparing the Cherenkov radiation pattern described by the digital image to the simulated Cherenkov pattern;
acquiring, by the processor of the computer, threshold data, which describes a predetermined threshold for the pattern comparison result; and
determining, by the processor of the computer, treatment apparatus control data based on the pattern comparison data and the threshold data, wherein the treatment apparatus control data is determined by the processor of the computer by comparing the pattern comparison result to the predetermined threshold and wherein the treatment apparatus control data describes a control signal to be issued to the radiation treatment apparatus for controlling an operation of the associated radiation treatment apparatus.

13. (canceled)

14. (canceled)

15. (canceled)

16. (canceled)

17. A medical system comprising:
a computer comprising a processor and a data storage device operatively coupled with the processor; and
a radiation treatment apparatus for carrying out radiation treatment on a patient,
wherein the computer is operably coupled with the radiation treatment apparatus for issuing a control signal to the radiation treatment apparatus for controlling the operation of the radiation treatment apparatus based on treatment apparatus control data,
wherein the data storage device stores instructions thereon that, when the instructions are executed by the processor of the associated computer, cause the computer to perform a method of determining control data for controlling the radiation treatment apparatus, the method comprising:
acquiring, by the processor of the computer, radiation pattern image data, which describes a digital image describing a Cherenkov radiation pattern on an anatomical body part of patient taken with an imaging device, a relative position between the imaging device and the radiation treatment apparatus being predetermined;
acquiring, by the processor of the computer, simulated radiation pattern data, which describes a simulated Cherenkov radiation pattern on the anatomical body part which has been simulated based on a planning image of the anatomical body part;
determining, by the processor of the computer, pattern comparison data is based on the radiation pattern image data and the simulated radiation pattern data, wherein the pattern comparison data describes a pattern comparison result describing a result of comparing the Cherenkov radiation pattern described by the digital image to the simulated Cherenkov pattern;
acquiring, by the processor of the computer, threshold data, which describes a predetermined threshold for the pattern comparison result; and
determining, by the processor of the computer, treatment apparatus control data based on the pattern comparison data and the threshold data, wherein the treatment apparatus control data is determined by the processor of the computer by comparing the pattern comparison result to the predetermined threshold and wherein the treatment apparatus control data describes the control signal to be issued to the radiation treatment apparatus for controlling an operation of the radiation treatment apparatus.
